(19) United States
(12) Patent Application Publication
(10) Pub. No.: US 2016/0331575 A1
(43) Pub. Date: Nov. 17, 2016

(54) DEVICE FOR FACILITATING NASAL BREATHING FOR SNORERS

(71) Applicant: Michel ALGLAVE, Saint-Andre (FR)

(72) Inventor: Michel ALGLAVE, Saint-Andre (FR)

(21) Appl. No.: 15/111,327

(22) PCT Filed: Jan. 14, 2015
(86) PCT No.: PCT/EP2015/051283

§ 371 Date: Jul. 13, 2016

(30) Foreign Application Priority Data
Jan. 22, 2014 (FR) 1450518

(51) Int. Cl. A61F 5/56; A61M 29/00

(52) U.S. Cl. CPC A61F 5/566; A61M 29/00

(57) ABSTRACT

A device for facilitating nasal breathing with the mouth closed, including a mouth guard having a maxillary support face and symmetrical two half-arcs on either side of a medial direction (m) and joining one another at a front area of the mouth guard. The mouth guard includes, in its front area an upper anterior edge, wherein it includes a lip protection including an upper part arranged in the extension of the upper anterior edge and extending above the maxillary support face. The upper part including two upper extensions configured so as to form a support area and advantageously to push forward the gingivolabial groove of a user enabling the airway to be increased.

### FIELD OF THE INVENTION

The invention relates to a device for facilitating nasal breathing in snorers. This device is intended to improve nasal breathing in snorers so as to limit or even prevent snoring. The device will be applicable in the treatment mainly of nasal snoring. The device is designed to be used with the mouth closed.

### PRIOR ART

Snoring is the breathing noise produced by a sleeping person. This noise is the vibration of throat tissue relaxed by sleep. During sleep, mouth breathing causes a double movement of opening and pulling back the lower jaw, narrowing the airway in the throat and causing vibration of the soft palate. Mouth breathing is in particular due to a nasal obstruction. The habit of mouth breathing becomes a reflex that is impossible to control.

There are numerous devices intended to reduce the snoring of the sleeper. These devices are all associated with mandibular advancement splints. These medical devices make it possible to hold the mandible, the lower jaw, in the forward position during sleep. This mechanical advancement frees the airway at the pharynx and reduces the phenomenon of obstruction of the airway at the back of the throat. These devices are comprised of an upper mouth guard positioned on the lower dental arch. The two mouth guards are connected by rods positioned on the outer faces of the mouth guards so as to return the lower jaw to its forward position.

These devices are relatively difficult to position properly in order to obtain satisfactory efficacy, in particular the mouth guards must be conformed by the user on each use. These devices are particularly irritating and users have difficulties keeping them in their mouth, especially if their nasal breathing is obstructed. These devices therefore are not properly used and the results are not satisfactory. Moreover, these devices do not treat the nasal causes of snoring.

There is therefore a need to provide a device for reducing or limiting snoring, which overcomes at least some of the disadvantages of the devices of the prior art.

### SUMMARY OF THE INVENTION

To this end, the invention relates to a device for facilitating nasal breathing. This device includes a mouth guard having a maxillary support face and two half-arcs, symmetrical on either side of a medial direction, joining one another at a front area of the mouth guard, and the mouth guard includes, in its front area, an upper anterior edge. The device is characterized in that it includes a lip protection including an upper part arranged in the extension of the upper anterior edge and extending above the maxillary support face, and said upper part includes two upper extensions configured so as to form an area for supporting the gingivolabial groove.

The device enables nostril expansion facilitating the nasal breathing of the user, thus limiting mouth breathing and therefore snoring.

The device according to the invention has the advantage of being easy to use and treating a nasal cause of snoring.

### BRIEF DESCRIPTION OF THE FIGURES

The drawings are provided as examples and do not limit the invention. They are schematic representations of a principle and intended for facilitating comprehension of the invention, and are not necessarily drawn to scale for practical use.

FIG. 1 shows a front perspective view of a first embodiment of the invention.

FIG. 2 shows a rear perspective view of a first embodiment of the invention.

FIG. 3 shows a front perspective view of the device according to the first embodiment of the invention, illustrating the relative positioning of the nose and nostrils of the user.

FIG. 4 shows a perspective view according to section AA of FIG. 3.

FIG. 5 shows a front perspective view of a second embodiment of the invention with the removable lip protection.

FIG. 6 shows a perspective view according to section BB—of FIG. 5.

FIG. 7 shows a schematic section view of a device according to the invention positioned in an oral cavity.

FIGS. 8 to 12 show a device according to the invention in three dimensions according to the same embodiment.

### DETAILED DESCRIPTION

Before a detailed review of embodiments of the invention is undertaken, optional features that may be used in association or as alternatives will described below.

It is first noted that the invention relates to a device for facilitating nasal breathing with the mouth closed, including a mouth guard having a maxillary support face and two symmetrical half-arcs on either side of a medial direction and joining one another in a front area of the mouth guard, the mouth guard including, in its front area, an upper anterior edge characterized in that it includes a lip protection including an upper part arranged in the extension of the upper anterior edge and extending above the maxillary support face, said upper part including two upper extensions configured so as to form an area for support of the upper gingivolabial groove of a user enabling the airway in the nose to be enlarged.

Advantageously, the lip protection is convex in a sagittal plane and in a frontal plane.

Advantageously, the convexity of the lip protection is configured so as to be capable of pushing the upper gingivolabial groove forward along a sagittal plane.

Advantageously, the lip protection includes a lower part arranged in the extension of the upper anterior edge, extending below the maxillary support face.

Advantageously, the two upper extensions of the lip protection are separated by a curve configured so as to cooperate with the upper gingivonasal groove.

Advantageously, the device includes a bead arranged on the periphery of the upper part of the lip protection.

Advantageously, the mouth guard has a flat mandibular support face opposite the maxillary support face.

Advantageously, the maxillary support face and/or the mandibular support face comprise(s), at a distal lateral area of each half-arc, a dished surface.

Advantageously, the device includes a lower palate edge extending from a front area behind the maxillary support face, said palate edge being formed by a substantially curved layer, the convexity of which is directed toward the mouth guard.

Advantageously, the device is formed symmetrically according to a sagittal plane passing through the medial direction.

Advantageously, at least one portion of the lip protection is removable so as to be attached by quick attachment means on the upper anterior edge of the mouth guard.

Advantageously, the lower palate edge includes at least two added-thickness areas formed on either side of the sagittal plane so as to be in contact with the lower gum at the lower incisors.

Advantageously, the lip protection has a thickness at the peaks of the extensions of less than or equal to 1 cm and a height extending between the maxillary support face and the peak of the extensions of between 23 and 30 mm.

Advantageously, the mandibular support face includes at least one longitudinal groove intended to receive the lower teeth by their crests.

Advantageously, the lip protection has a variation in its hardness.

Advantageously, the lip protection has a decrease in hardness from the maxillary support face toward the two extensions.

Advantageously, the lip protection has a decrease in thickness from the back to the front.

Advantageously, the two extensions do not extend beyond the width of the user's nose at the nostrils.

In the following description, unless otherwise indicated, the term symmetrical means that the device of the invention advantageously has a symmetry, either for certain portions along an axis in the medial direction, which will be explained below, or in a sagittal plane. The term symmetrical does not mean that slight dimensional or geometric variations are impossible around a symmetry, strictly speaking. In particular, according to the corrections to be made to the user's morphology, the device may have slight variations between the left and right lateral portions of the invention capable of cooperating with the dental arches. The term “sagittal plane” refers to its usual anatomical definition as a vertical plane perpendicular to the frontal plane. Typically, this plane is parallel to an anteroposterior axis of the head and passes through a point located halfway between the two eyes. In the invention, the medial direction is included in the sagittal plane, the sagittal plane being capable of being defined as the plane containing said medial direction and substantially perpendicular to the maxillary support face, which will be described in greater detail below. The adjective “frontal” is used here to refer to a direction of intersection between a frontal plane and the sagittal plane, i.e. generally a vertical direction.

Terms such as under and below refer to a position relative to a vertical direction generally according to the thickness of the mouth guard.

The term dental arch sector refers to an anatomical portion of the user capable of including a bony portion and/or a dental portion to which the device may be applied.

The adjective “maxillary” refers to the upper jaw and the adjective “mandibular” refers to the lower jaw.

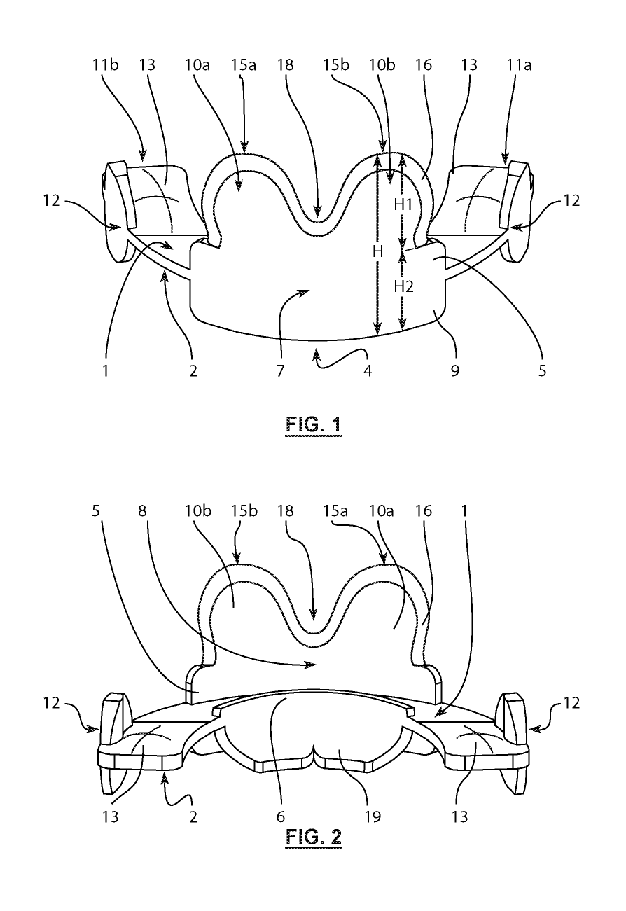

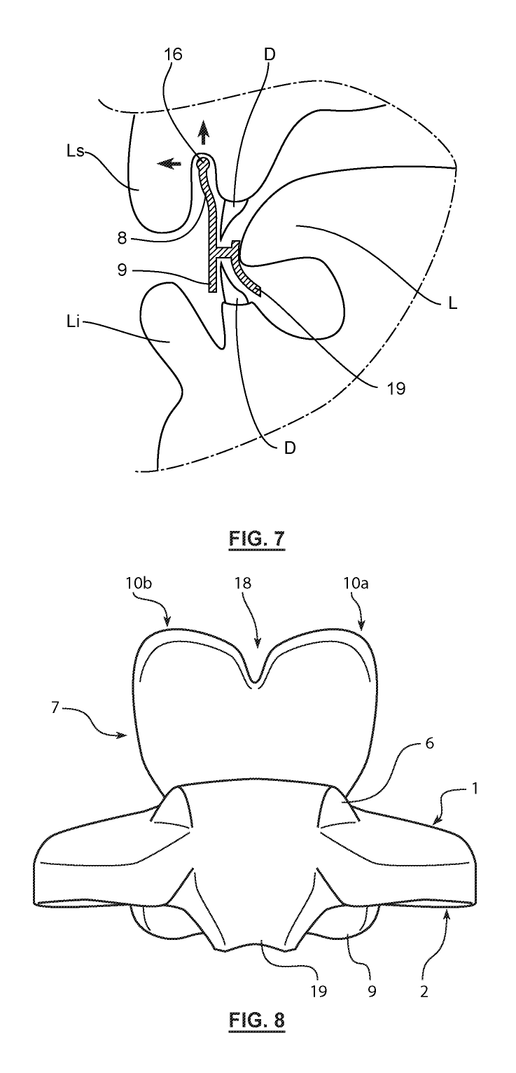

The device according to the invention is presented in different views in FIGS. 1 to 7.

It generally has a shape matching the contours of the upper dental arch of the user intended to receive the device.

As with known mandibular advancement devices, the device of the invention comprises a mouth guard enabling at least upper jaw support. To this end, the mouth guard includes a maxillary support face 1, visible for example in perspective in FIG. 2.

Advantageously, but not exclusively, the maxillary support face 1 is substantially flat aside from a distal lateral area 11a, 11b, which will be explained below. The maxillary support face 1 and its dished surface 13 enables the teeth of the upper dental arch of the user to be applied.

Opposite the maxillary support face 1, the mouth guard preferably has a mandibular support face 2, which may have a configuration substantially identical to the maxillary support face 1 except that it is preferably flat while the maxillary face 1 advantageously has dished surfaces 13 presented in detail below.

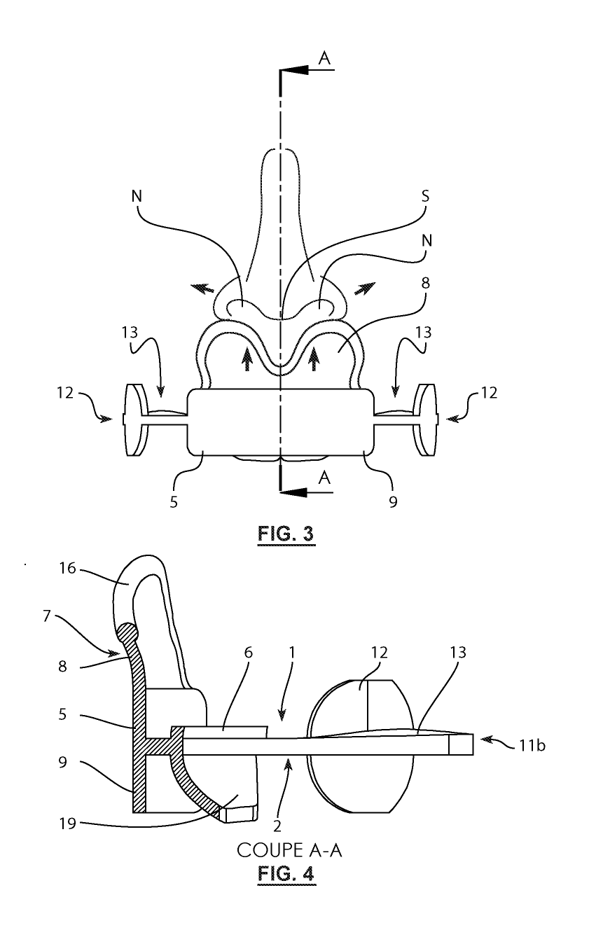

In general, to follow the anatomical contour of the upper dental arch of the user, the mouth guard has two half-arcs 3a, 3b, each placed on one side of the mouth guard around a front area 4 forming the junction between the two symmetrical half-arcs 3a, 3b. The axis of symmetry of the two half-arcs 3a, 3b corresponds to a medial direction m of the mouth guard, which cuts it into two equal portions. This direction “m” is visible in particular in the front view, FIG. 3, and corresponds to the intersection between the sagittal plane and a substantially horizontal plane in which at least the maxillary support face 1 is approximately located.

It is easily understood that the shape and size of the mouth guard are adapted to the morphology of the user and may therefore be variable. Consequently, the invention is thus available in a number of sizes. The mouth guard is advantageously flexible and therefore resiliently deformable as indicated above, and may be made of different biocompatible materials and particularly polyvinyl chloride (PVC) or silicone.

In the portion with the maxillary support face 1 and the mandibular support face 2, the mouth guard forms a bite plane located between the top arch and the bottom arch. Its typical thickness is approximately 2 to 4 millimeters.

The mouth guard includes an upper anterior edge 5 and advantageously an upper posterior edge 6. These edges 5-6 are positioned in the front area 4 of the mouth guard, above the maxillary support face 1. The anterior edge 5 is the edge arranged outside the concavity of the mouth guard, while the posterior edge 6 is the edge arranged inside the concavity of the mouth guard. Advantageously, the upper anterior 5 and posterior 6 edges follow the convexity of the mouth guard. The edges extend substantially in a plane perpendicular to the plane of the maxillary support face. These edges 5-6 form anterior and posterior guides for the maxillary dental arch, in particular for guiding the teeth into the mouth guard.

According to the invention, aside from the mouth guard, the device includes means making it possible, under a thrust exerted by the user's tongue and a clamping of the mouth guard between the upper jaw and the lower jaw, to transfer said forces at the gingivolabial groove. The term gingivolabial groove refers to the upper groove, i.e. the maxillary groove. More specifically, gingivolabial groove refers to the space located between the upper lip and the upper gum and including at least the upper labial frenulum.

To perform the desired action, the device includes a lip protection 7, embodiments of which may be seen in FIGS. 1 to 7.

The lip protection 7 is located in the anterior are of the device of the invention and forms the frontal portion of the assembly. Its shape is advantageously convex. Advantageously, the convexity is produced not only in planes parallel to the sagittal plane but also in perpendicular planes. The lip protection 7 has a convexity in the sagittal plane and in the frontal plane. Said convexity of the lip protection 7, more specifically said two convexities, enable the gingivolabial groove to be pushed forward according to a sagittal plane. The lip protection 7 is arranged in the extension of the upper anterior edge 5. Preferably, the lip protection 7 includes an upper part 8 extending from the upper anterior edge 5 and above the maxillary support face 1. According to the invention, the upper part 8 of the lip protection 7 includes two upper extensions 10a, 10b extending to the upper gingivolabial groove opposite the nostrils. The V shape, i.e. producing a progressive distancing of the extensions toward their distal ends 15a-15b, is designed to respect the upper labial frenulum. When the user clamps his or her teeth, said two extensions 10a, 10b will improve the nasal breathing of the user. The two extensions 10a, 10b simultaneously exert a forward thrust force in a sagittal plane, a vertical upward thrust force and a lateral thrust force, substantially obliquely away in a frontal plane. This combination of thrust forces causes a modification of the nose improving nasal breathing. The airway in the nose is enlarged. More specifically, said thrust forces modify the shape of the nostrils. Similarly, said thrust forces modify the nasal passages and in particular the valve of each passage, improving nasal breathing. In general terms, there is a dilation of the nostrils. Advantageously, the diameter of the nostril orifices is increased transversally, enabling better nasal breathing. This is particularly beneficial because most snorers have a problem of deficient nasal breathing. The two extensions 10a, 10b are separated by a curved portion 18, the shape of which is preferably configured so as to provide a space for the labial frenulum and be non-damaging. The curved portion forms a bottom point at the sagittal plane. The top of the upper part 8 at the curved portion 18 is lower than the height of said part at the distal ends 15a, 15b of the two extensions 10a, 10b.

Preferably, the lip protection 7 includes a lower part 9 extending from the upper anterior edge 5 and extending below the maxillary support face 1.

The lip protection 7 advantageously encompasses the lower part 9, the upper anterior edge 5 and the upper part 8 with said two extensions 10a-10b.

As a non-limiting example, the height of the upper anterior edge 5 is between 5 mm and 10 mm, preferably on the order of 7 mm to 9 mm, the height of the upper posterior edge 6 is between 2 and 6 mm. Preferably, the height H of the lip protection 7 is between 30 and 40 mm, preferably between 34 mm and 35 mm. The height h1 of the upper part 8 is advantageously equal to the height h2 combining the upper anterior edge 5 and the lower part 9, preferably between 15 mm and 20 mm, and preferably 17 mm. According to an advantageous embodiment it is possible for the height h1 to be capable of reaching 30 mm, for example 27 mm.

The lip protection 7 has, for example, a length of 20 to 50 mm. Preferably, the two extensions 10a, 10b extend over a length of 15 to 45 mm. Said length of the two extensions 10a, 10b is, for example, configured so as to correspond to the width of the nose of the user at the nostrils. Preferably, the two extensions 10a, 10b do not extend above the frontal portion of the device. The frontal portion is the portion of the device intended to be arranged opposite the user's nose. Advantageously, the two extensions 10a, 10b extend on either side of the upper labial frenulum over a total length corresponding to the width of the user's nose.

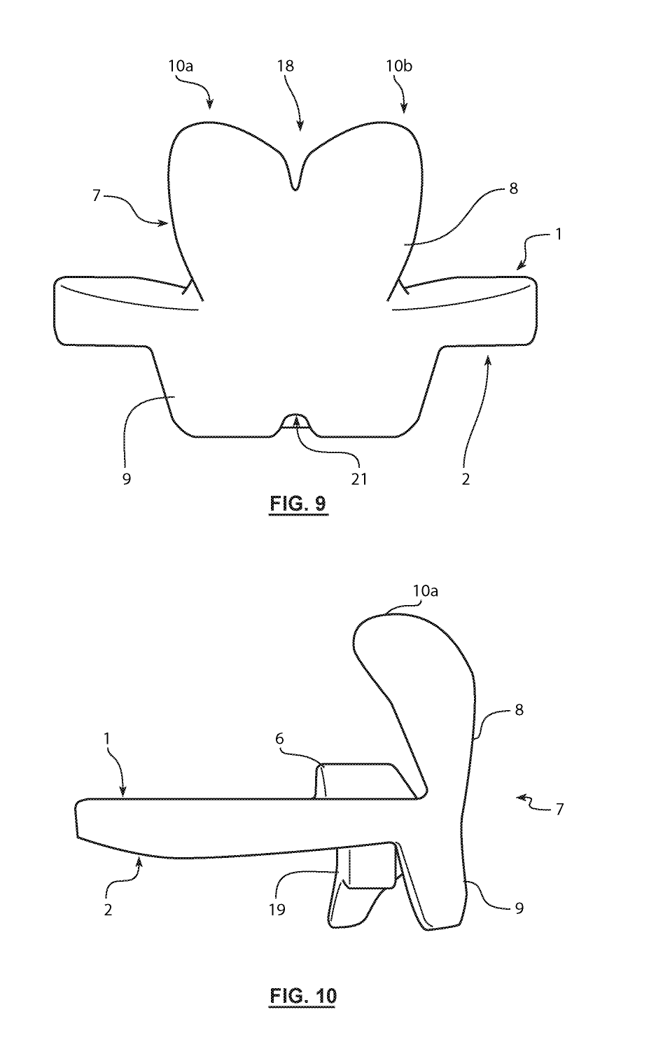

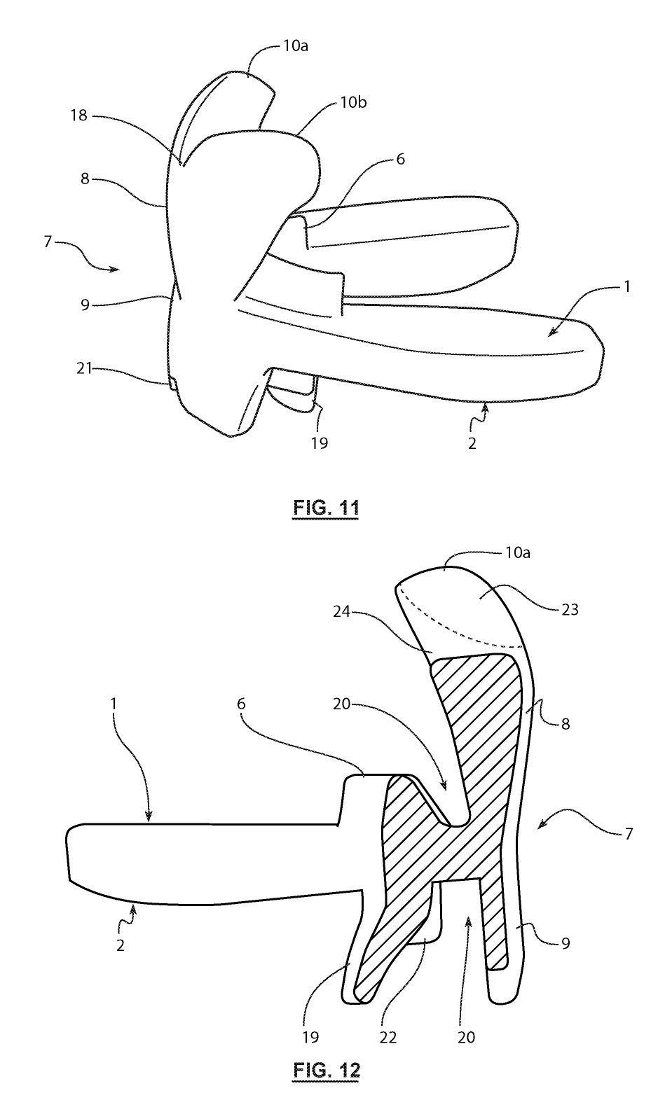

As an example shown in FIGS. 8 to 12, the extensions 10a, 10b have a thickness at their peak less than or equal to 1 cm.

The lip protection 7 is intended to be in contact by its outer face with the user's upper and lower lips. Preferably, the upper part 8 is intended to be in contact with the upper lip Ls while the lower part 9 is intended to be in contact with the lower lip Li.

The convex shape of the lip protection 7 is also intended to be non-damaging for the user who must wear said device over long periods of time, in particular for the entire night. The lip protection 7 is configured so as to perfectly fit the outer shape of the upper dental arch. To this end, it is preferable that the lip protection 7 be equipped at its periphery, preferably upper, intended to be in contact with the gingivolabial groove and more specifically the base of the nostrils, with a bead 16. Said bead 16, with a substantially round cross-section, makes it possible to prevent injury and/or irritation of the mouth tissue. Said bead 16 advantageously has more flexibility than the rest of the device. Preferably, the bead 16 is made of the same material as the rest of the device. It forms an edge with a broader cross-section than the thickness of the rest of the lip protection 7.

According to an alternative tending to improve the efficacy and comfort of the device according to the invention, the lip protection 7 is advantageously designed to have a hardness that is variable over its surface. More specifically, it is preferably for the hardness of the lip protection 7 to decrease from the maxillary face 1 to the extensions 10a, 10b over at least a portion of its thickness. Thus, the hardness of the lip protection 7 is preferably lower at the extensions 10a, 10b and higher at the connection with the maxillary face 1. The variation may be progressive or not, with at least two portions of different hardness. This variation over the height of the lip protection 7, more specifically the height h2 described above, ensures a transmission of the forward-moving force generated at the maxillary face 1 to the extensions 10a, 10b, while ensuring a forward movement of the peak of the extensions 10a, 10b improving the dilation of the nostrils without being damaging to tissue. The hardest portion in fact transmits the forces better, while the less hard portion is more easily deformable at areas of lower stress.

Similarly, it is preferable for the hardness of the extensions 10a, 10b to be variable in the direction parallel to the sagittal plane. Thus, the hardness of the extensions 10a, 10b is greater at the back face and lower at the front face, ensuring mobility of the extensions and satisfactory transmission of the forward-moving forces.

For example, the hardness of the lip protection 7 and more specifically of the extensions 10a, 10b is 80 shore A in the areas of high hardness 24 and 60 shore A in areas of low hardness 23.

To enhance the efficacy of the device of the invention, it advantageously has a particular configuration for the portion where the upper molars are to be applied. More specifically, the maxillary support face 1 preferably has a dished profile at an area for application of at least one molar tooth. As shown, for example, in FIG. 4, this particular configuration is produced at two distal lateral areas 11a, 11b located substantially at the distal ends of each half-arc 3a, 3b. The dished profile advantageously has a maximum height toward the middle of the width of the maxillary support face 1. It may also be dished in the direction transversal to the length of the maxillary support face 1 with an elevation that reaches a maximum toward the middle of the first upper molar, before the distal end of each half-arc 3a, 3b.

Additionally, or alternatively, the thickness of the mouth guard may be variable and in particular increase toward the distal end of each half-arc 3a, 3b at the distal areas 11a, 11b. By increasing the thickness of the mouth guard in its distal areas 11a, 11b, for example, the dental support area of the maxillary support face 1 is slightly raised with respect to the rest of the mouth guard so as to form the dished surface 13. The dished surface 13 may also be arranged on the mandibular support face 2 or simultaneously on both maxillary 1 and mandibular 2 support faces. The added thickness due to the dished surface 13 causes a lowering of the mandible. The joints are relaxed, facilitating a forward movement of the mandible in a sagittal plane. This forward movement makes it possible to substantially place the maxillary dental arch in line with the mandibular dental arch, helping to limit snoring.

According to another possibility of the invention, the distal lateral areas 11a, 11b have parts, preferably outer distal parts 12a, 12b preferably for each half arc 3a, 3b. Advantageously, the device does not include inner distal parts. Said parts 12a, 12b are in external contact with a molar tooth and particularly the first upper molar so as to exert a lateral bearing force on the latter. This configuration of parts makes it possible to properly affix the device without exerting any significant force on the teeth. The parts 12a, 12b preferably have a profile according to a cross-section perpendicular to the sagittal plane with enlarged portions progressively returning above the maxillary support face 1. The parts 12a, 12b extend substantially perpendicularly with respect to the plane of the maxillary support face 1 and preferably above and below said face.

On the face opposite the maxillary support face 1, the mandibular support face 2 however advantageously has at least one edge projecting about said mandibular support face 2.

Preferably, the mandibular support face 2 includes at least one longitudinal groove.

Said longitudinal groove extends advantageously over the length of the lip protection 7. The longitudinal groove is shaped so as to receive the crests of the lower teeth, more specifically incisors and canines. The groove thus forms a predefined position of mandibular forward movement helping the user to find a satisfactory forward position while maintaining the forward position. According to a preferred embodiment, the device includes at least two parallel longitudinal grooves. The grooves define different mandibular forward-movement positions enabling a progression of the forward movement. The user will first use the groove producing the smallest forward movement, then progressively will use the groove producing the greatest forward movement. Advantageously, the device includes a lower palate edge 19 capable of forming an area of space in which the distal end of the tongue may be received and makes it possible to guide the movement of the lower incisors during movements of the mandible. The lower palate edge 19 extends from the upper posterior edge 6 of the mouth guard. The palate edge 19 is formed by a substantially curved layer, the convexity of which is oriented toward the mouth guard. The lower palate edge 19 advantageously has the shape of a sphere or hemisphere sector over an angular portion, for example, less than one-quarter sphere. The lower palate edge 19 is primarily configured so as to guide the tip of the tongue forward and the top in a sagittal plane. The palate edge 19 accompanies the tip of the tongue limiting its accumulation at the back of the mouth and thereby limiting snoring. In addition, the lower palate edge 19 forms a guide for positioning teeth of the mandibular dental arch and in particular incisors in the mouth guard at the mandibular support face. Advantageously, the lower palate edge 19 includes at least two areas so-called frontal added thickness intended to ensure contact with the lower gum, more specifically at the lower incisors so as to limit or even reduce direct contact with the teeth. The added thicknesses enable an approach on the tongue side of the mandibular bone. The bone commonly has a torus that is understood to be a harder bone than the surrounding bone. This makes it possible to prevent vestibulation of the incisors, that is, a forward movement toward the front of the incisors. Said added thicknesses are advantageously arranged symmetrically on either side of the sagittal plane. According to a preferred option, the width of the mouth guard 20 at the mandibular face 2 is greater than the width of said mouth guard 20 at the maxillary face 1. More specifically, the lower palate edge 19 is retracted with respect to the upper posterior edge, and the lower part 9 is in line with the upper part 8 of the lip protection 7.

The lower palate edge 19 extends preferably over a narrower angular sector than the angular sector over which the lip protection 7 extends. Preferably, the lower palate edge 19 extends over an angular sector on the order of 30° of the mouth guard.

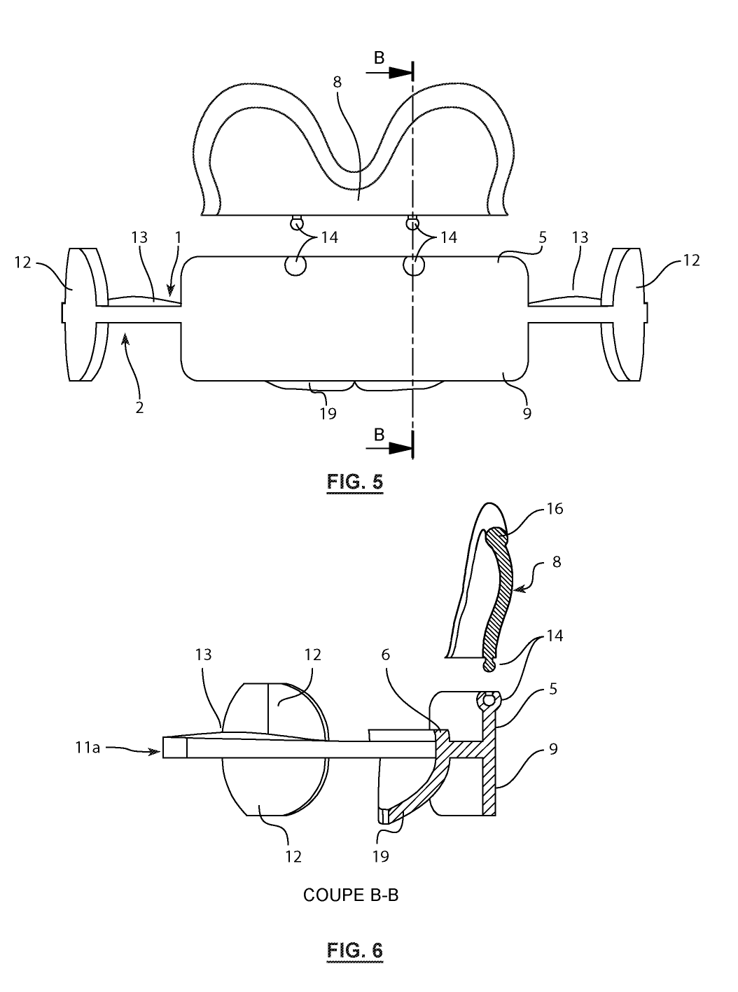

According to an embodiment shown in FIGS. 5 and 6, the upper part 8 of the lip protection 7 is at least partially removable. The device includes means 14 for quick attachment 14 of the upper part 8 of the lip protection 7 on the mouth guard and preferably on the upper anterior edge 5. These quick attachment means make it possible to attach an upper part 8 and therefore form a lip protection 7 as described above in order to facilitate nasal breathing on a base mouth guard for example on a conventional orthodontic apparatus. As a non-limiting example, these quick attachment means 14 are, for example, male-female systems such as notches and complementary projections on the upper anterior edge 5 and on the upper part 8. It is thus possible to form a device base portion capable of selectively cooperating with one of several upper parts 8 in particular for a simple adaptation to different user anatomies.

A procedure of the invention is described below.

The user's mandible, which clamps the teeth, is placed in the channel formed by the mouth guard at the maxillary support face 1 moved forward in order to obtain an advantageously end-to-end position of the upper and lower incisors. Advantageously, the mouth guard enters the user's mouth by being transversally squeezed between the thumb and the index finger.

First, it is possible to adjust the dimensions of the device to the morphology of the user and in particular to thermoform the mouth guard according to the desired transverse dimension according to the size of the user's palate.

When the teeth are clamped, the distal ends 15a, 15b of the extensions 10a-10b of the lip protection 7 will simultaneously exert a vertical thrust toward the base of the nostrils and a horizontal thrust in a direction parallel to the medial direction m oriented toward the front of the device. This combination of thrusts helps to move the upper portion of the upper lip away from the upper dental arch in particular by thus expanding the diameter of the nostrils.

The apparatus thus acts essentially on the effect of the user's reflexive contraction. Nevertheless, by simply clamping the teeth in the mouth guard 1, the user activates the device simply by means of its resiliency.

One may note the benefit in the device of having portions projecting above the maxillary 3 and mandibular 2 support faces, which are not continuous along the half-arcs 23a, 23b. In particular, in the example shown, mouth guard portions do not have any material other than the thickness of the mouth guard 1 between said faces 2, 3, for example between the parts 16a, 16b and the lip protection 7. Thus a wide range of possibilities for modifying the resilient deformation are maintained.

### REFERENCES

1. Maxillary support face

2. Mandibular support face

3. a-3.b Half-arc

4. Front area

5. Upper anterior edge

6. Upper posterior edge

7. Lip protection

8. Upper part

9. Lower part

10. a-10.b Upper extensions

11. a-11.b Distal lateral area

12. Outer distal part

13. Dished surface

14. Quick attachment means

15. a-15.b Distal ends

16. Bead

18. Curved portion

19. Lower palate edge

20. Mouth guard

21. Curved portion

22. Added thickness

23. Area of low hardness

24. Area of high hardness

m. Medial direction

Ls. Upper lip

Li. Lower lip

L. Tongue

S. V-shaped gingivolabial groove

N. Nostril

D. Tooth

H. Lip protection height

h1. Upper part height

h2. Height of upper anterior edge+lower part

1. Device for facilitating nasal breathing with the mouth closed, including a mouth guard having a maxillary support face and two symmetrical half-arcs on either side of a medial direction (m) and joining one another in a front area of the mouth guard, the mouth guard including, in its front area, an upper anterior edge wherein the mouth guard includes a convex lip protection in a sagittal plane and in a frontal plane including an upper part arranged in the extension of the upper anterior edge and extending above the maxillary support face, said upper part including two upper extensions extending over a length corresponding to the width of the nose of a user at the nostrils, separated by a curved portion configured so as to cooperate with the upper gingivolabial groove of the user and configured so as to form an upper gingivolabial groove of the user enabling the airway in the nose to be enlarged.

2. The device according to claim 1 in which the convexity of the lip protection is configured so as to push the upper gingivolabial groove forward in a sagittal plane.

3. The device according to claim 1 in which the lip protection includes a lower part arranged in the extension of the upper anterior edge extending below the maxillary support face.

4. The device according to claim 1 including a bead arranged on the periphery of the upper part of the lip protection.

5. The device according to claim 1 in which the mouth guard has a flat mandibular support face opposite the maxillary support face.

6. The device according to claim 1 including a lower palate edge extending from a front area behind the maxillary support face, said palate edge being formed by a substantially curved layer, the convexity of which is oriented toward the mouth guard.

7. The device according to claim 6 in which the lower palate edge includes at least two areas of added thickness formed on either side of the sagittal plane so as to be in contact with a lower gum at the lower incisors.

8. The device according to claim 1, formed symmetrically according to a sagittal plane passing through the medial direction (m).

9. The device according to claim 1 in which at least a portion of the lip protection is removable so as to be attached by a quick attachment system on the upper anterior edge of the mouth guard.

10. The device according to claim 1 in which the lip protection has a thickness at the peaks of the extensions less than or equal to 1 cm and a height extending between the maxillary support face and the peak of the extensions between 23 and 30 mm.

11. The device according to claim 1 in which the mandibular support face includes at least one longitudinal groove configured to receive the lower teeth by their crests.

12. The device according to claim 1 in which the lip protection has a variation in hardness.

13. The device according to claim 12 in which the lip protection has a decrease in hardness from the maxillary support face toward the two extensions.

14. The device according to claim 13 in which the lip protection has a decrease in hardness from the back to the front.

15. The device according to claim 1 in which the two extensions do not extend beyond the width of the user's nose at the nostrils.
